(19) United States
(12) Patent Application Publication
(10) Pub. No.: US 2009/0264829 A1
(43) Pub. Date: Oct. 22, 2009

(54) INJECTION SYRINGES

(75) Inventors: Nigel Harris, Cardiff (GB); Nicholas John Stratton Davis, Cardiff (GB)

Correspondence Address: KING & SCHICKLI, PLLC, 247 NORTH BROADWAY, LEXINGTON, KY, 40507, US

(73) Assignee: Frontier Plastics Limited

(21) Appl. No.: 11/721,836

(22) PCT Filed: Dec. 14, 2005
(86) PCT No.: PCT/GB05/04828

§ 371 Date: Jun. 15, 2007

(30) Foreign Application Priority Data
Dec. 16, 2004 (GB) 0427507.9

(51) Int. Cl. A61M 5/31; A61J 1/14

(52) U.S. Cl. 604/190; 604/416

(57) ABSTRACT

A sheath (1) is disclosed which is suitable for covering the needle of a syringe (2). The sheath (1) includes a filter (3) which projects partially from the end of the sheath to aid the drawing of liquid into the syringe. It is intended that the needle and/or syringe are sold fitted with the sheath (1) so that there is no need to expose the needle until the user is ready for injection. The invention includes a blister pack (12 FIG. 4) which includes a ready assembled syringe, a mixing pan and paraphernalia for the preparation of the solution to he injected.

It is generally accepted that a major health risk is associated with blood borne organisms being transferred between individuals either by accidental needlestick injury or by the sharing of needles in the intravenous drug using community. Consequently it has become common practice to provide drug users with free injection syringes and other equipment to enable them to carry out self injection of drugs in a safe way. One common method of achieving injection of drugs is to dissolve a powdered or solid form of the drug in a solution. The solution is then drawn into the syringe and subsequently injected. There is the risk that contaminants might be drawn into the syringe and then injected into the body of the user which could have serious consequences. To overcome this filters have been made available to prevent solid particles being drawn into the syringe. Such a filter is shown in WO 98/51356.

It is just one object of embodiments of this invention to provide equipment enabling drug users to self inject in a manner which overcomes difficulties experienced in the past.

Accordingly, this invention provides a sheath for covering the needle of a syringe, the sheath being removably connected, at one end, to the body of the syringe around the needle, the sheath also incorporating a filter fitted into or over the sheath's other end, the sheath being characterised in that the filter projects partially from the said other end.

With this arrangement the filter is fitted to the end of the syringe needle and so a drug solution can be drawn in to the syringe and any contaminants are blocked by the filter. Once the syringe has been filled, immediately prior to injection, the sheath can be removed and the drug solution administered.

The filter can be designed to filter out any desired material such as bacteria and viruses as well as solid particles. This invention represents a significant improvement to the current procedure in that the use of a filter becomes integral to the drawing up of the solution and further it is not necessary to expose the needle until the syringe is filled ready for injection. Moreover, if in certain embodiments the needle is located into the filter the liquid will be drawn through the filter directly into the needle. In the situation where the needle does not locate into the filter then the sheath will form a seal around the needle such that when the plunger is withdrawn a vacuum will be created inside the sheath causing the liquid to be drawn through the filter into the body of the syringe.

A sheathed needle and a syringe can be supplied separately. In this case the needle as supplied would be protected by the sheath, with the filter fitted to it, and when the needle is connected to the syringe it will operate in the same fashion as is described above.

The invention also extends to a mixing pan in the form of a dish with a flat base and a projecting handle, wherein the handle angles down from the top plane of the dish and terminates in a flat portion lying in the same plane as the flat base of the dish.

The special formation of the handle ensures that the mixing pan will not tend to tip during the process of withdrawing a prepared solution from the pan into the syringe.

The invention further extends to an injection kit comprising a removable filter for a syringe as hereinbefore defined, a mixing pan again as hereinbefore defined, and a quantity of acid to assist in dissolving a drug to be injected, all retained by a blister pack.

The kit could also include a phial of sterile water and a swab within the blister pack and any other paraphernalia as may prove necessary, e.g. a sterile mixing device and sterile gloves.

The invention may be performed in various ways and a preferred example thereof will now be described, with reference to the accompanying diagrammatic drawings, in which:

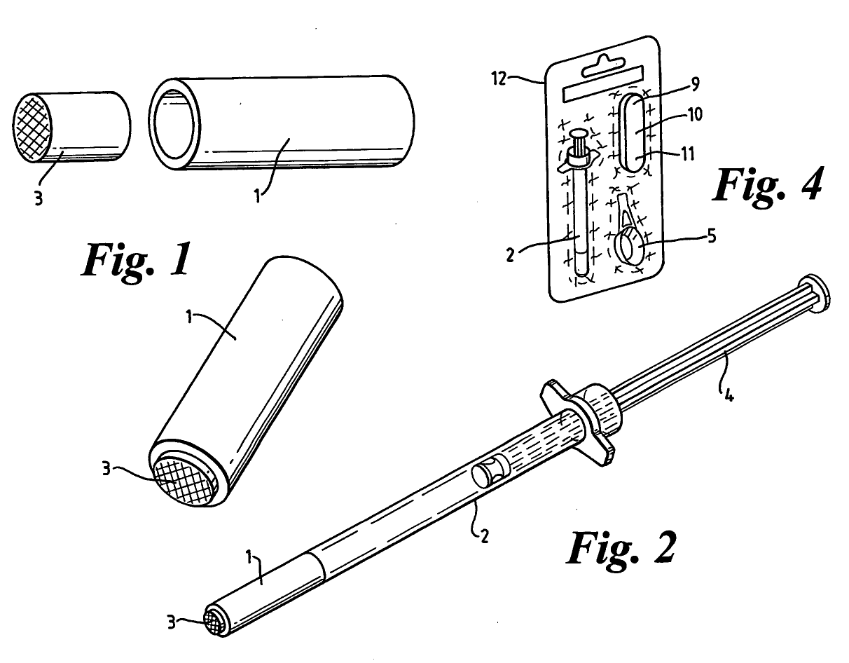

FIG. 1 is an enlarged view of a sleeve for a filter according to embodiments of this invention for use with an injection syringe;

FIG. 2 is a side view of a syringe with the sleeve fitted on the end;

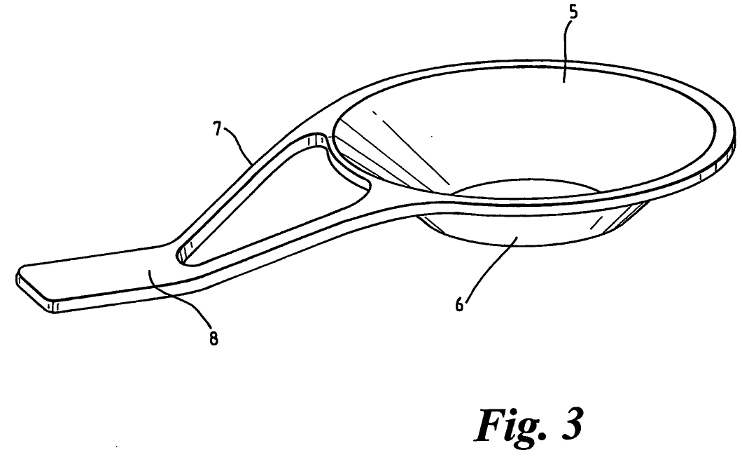

FIG. 3 is a perspective view of a mixing pan for use with the syringe; and

FIG. 4 shows a blister pack for packaging the articles shown in FIGS. 1, 2 and 3.

The device shown in FIG. 1 comprises a rigid plastic sleeve 1 which can be slid tightly over the end of a syringe 2, as shown in FIG. 2. In this instance the sleeve is a cylinder, having constant external and internal diameters. A filter plug 3 fitted tightly into the sleeve 1 will receive the tip of the exposed needle of the syringe 2 so that the needle projects into the filter plug. The filter projects from the end of the sleeve so that liquid may be drawn easily into the syringe. The filter is in the form of a plug of fibrous material. When the plunger 4 of the syringe is withdrawn a drug solution can then be drawn into the syringe, through the needle, after passing through the filter 3. The filter blocks any unwanted contaminant.

The solution to be drawn into the syringe can be prepared in the mixing pan, as shown in FIG. 3. This pan 5 has a flat base 6 and a projecting handle 7 which terminates in a flat portion 8 which is in the same plane as the flat base 6 of the dish. Then when materials are being mixed in the dish and when the solution is being withdrawn from the dish into a syringe the dish can be kept stable by holding down on the flat portion 8 of the handle 7. The portion of the filter projecting from the sleeve allows all the liquid in the pan to be drawn into the syringe.

It is preferred that the sleeve and filter will be fitted to the syringe and sold to the end user ready assembled. In that instance the user does not have to fit the sleeve and filter to the syringe and therefore there is far less risk of contaminating or blunting the syringe needle.

The filter plug 3 may have any desired properties such as anti-microbial properties in order to remove harmful bacteria, yeasts, fungi and viruses. For example an electrostatically charged filter may be employed to hold contaminants in the filter.

FIG. 4 shows a blister pack 12 which holds in kit form the pre-assembled syringe 2, sleeve 1 and filter 3, together with the mixing pan 5 and a quantity of acid 9 for use in preparing appropriate injectable drugs. Other elements may be included in the kit e.g. a sterile swab 10 and sterile water 11. The blister pack 12 allows the user to purchase all the elements necessary for drug preparation so they will then be less likely to use non-sterile equipment.

In the embodiment described the plug 3 is not intended to be removable from the sheath. However, the filter may be removable and, indeed, the fact that the filter projects from the sheath helps to accommodate replacement which may take place e.g. for replacement by a filter having either the same or different properties. Similarly, the sheath shown is a plane cylinder, however, the sheath and syringe may be of other complementary shapes to allow connection. Further, in other embodiments the sheath may be attached to the syringe, or to a needle holder of the syringe, by a breakable seal or other frangible connection.

1. A sheath for covering the needle of a syringe, the sheath being removably connected, at one end, to the body of the syringe around the needle, the sheath also incorporating a filter fitted into or over the sheath's other end, the sheath being characterised in that the filter projects partially from the said other end.

2. A sheath as claimed in claim 1 wherein the tip of the needle is located in the filter.

3. A sheath as claimed in claim 1 wherein the sheath is in the form of a cylinder and the filter is a plug of material which is fitted partially into the internal diameter of the cylinder and projects partially therefrom.

4. A sheath as claimed in claim 1 wherein the filter enables the filtering of organisms.

5. A syringe needle covered by a sheath as claimed in claim 1.

6. A syringe having a needle covered by a sheath as claimed in claim 1.

7. A mixing pan in the form of a dish with a flat base and a projecting handle, wherein the handle angles down from the top plane of the dish and terminates in a substantially flat portion lying in the same plane as the substantially flat base of the dish.

8. An injection kit comprising an assembled syringe, needle and sheath being removably connected, at one end, to the body of the syringe around the needle, the sheath also incorporating a filter fitted into or over the sheath's other end, the sheath being characterised in that the filter projects partially from the said other end, a mixing pan in the form of a dish with a flat base and a projecting handle, wherein the handle angles down from the top plane of the dish and terminates in a substantially flat portion lying in the same plane as the substantially flat base of the dish and a quantity of acid to assist in dissolving a drug to be injected, all retained by a blister pack.

9. A kit according to claim 8, including a phial of sterile water and a swab within the blister pack.

10. (canceled)

11. An injection kit as claimed in claim 8 wherein the tip of the needle is located in the filter.

12. An injection kit as claimed in claim 8 wherein the sheath is in the form of a cylinder and the filter is a plug of material which is fitted partially into the internal diameter of the cylinder and projects partially therefrom.

13. An injection kit as claimed in claim 8 wherein the filter enables the filtering of organisms.

14. A sheath as claimed in claim 2 wherein the sheath is in the form of a cylinder and the filter is a plug of material which is fitted partially into the internal diameter of the cylinder and projects partially therefrom.

15. A sheath as claimed in claim 2 wherein the filter enables the filtering of organisms.

16. A sheath as claimed in claim 3 wherein the filter enables the filtering of organisms.

17. A syringe needle covered by a sheath as claimed in claim 2.

18. A syringe needle covered by a sheath as claimed in claim 3.

19. A syringe needle covered by a sheath as claimed in claim 4.

20. A syringe having a needle covered by a sheath as claimed in claim 2.

21. A syringe having a needle covered by a sheath as claimed in claim 3.
